(19) United States
(12) Patent Application Publication
(10) Pub. No.: US 2010/0114158 A1
(43) Pub. Date: May 6, 2010

(54) DELIVERY ASSEMBLY, DELIVERY TIP, AND METHOD OF USING SAME

(75) Inventors: Paul Hattan, Minneapolis, MN (US); John O. White, Lakeville, MN (US); Jason Gerold, Shakopee, MN (US)

Correspondence Address: Casimir Jones S.C., 440 Science Drive, Suite 203, Madison, WI, 53711, US

(73) Assignee: NERITES CORPORATION, Madison, WI (US)

(21) Appl. No.: 12/568,547

(22) Filed: Sep. 28, 2009

Related U.S. Application Data
(60) Provisional application No. 61/100,781, filed on Sep. 29, 2008.

(51) Int. Cl. A61B 17/03; A61M 35/00

(52) U.S. Cl. 606/214; 604/82

(57) ABSTRACT

Embodiments of a delivery device, a kit comprising the delivery device, and methods of using the same are presented. In one embodiment, the delivery device includes a syringe assembly defining two or more reservoirs each configured to hold a dispensable material. Additionally, the delivery device may include a delivery tip. The delivery tip may have an inlet, an outlet, a central flow-restricting portion, and one or more channels adjacent to the central flow-restricting portion that provide fluid communication between the inlet and the outlet, the one or more channels including a section that tapers inwardly toward the outlet. In one embodiment, the delivery device is configured such that if the two or more reservoirs hold dispensable material then the syringe assembly can be actuated to dispense the dispensable material from the two or more reservoirs substantially simultaneously into the inlet.

### CROSS-REFERENCE TO RELATED APPLICATIONS

This application claims the benefit of U.S. Provisional Application No. 61/100,781 filed Sep. 26, 2008, entitled “Delivery Assembly, Delivery Tip, and Method of Using Same” by Paul Hattan, Jason Gerold and John O. White the entire contents of which is specifically incorporated herein by reference without disclaimer.

### BACKGROUND OF THE INVENTION

1. Field of the Invention

The present invention relates to multi-component spray delivery devices, kits, and methods.

2. Description of the Related Art

Current applicators for, e.g., two-component delivery systems, particularly for use with bioadhesives are simple, minimalist products that are little more than an oversized syringe. Unfortunately, manual actuation of the dispensing mechanism in existing applicators often dispenses uneven amounts of liquids, particularly over a short time period.

Many scientific and industrial applications require a well-atomized liquid spray. Liquid atomization is most readily achieved by shearing the liquid with a gas at or before the spray discharge (air-assist atomization; air brushes). In some applications, however, the introduction of air is undesirable or impractical. In such cases, atomization is achieved by liquid-liquid shearing caused by velocity gradients near the discharge orifice. Existing commercial spray nozzles utilize radial fluid channels to direct fluid from an upstream, outer annulus towards the centrally located discharge orifice. To induce rotation and subsequent shearing and atomization, the feed channels on existing commercial spray nozzles are offset from center, or may be curved in a swirl pattern, and/or may include curved guide vanes present near the orifice.

Several scientific, industrial and medical applications require the spray of a two-component adhesive. In these applications, the component liquids are typically mixed within the spray nozzle then discharged together. The time between mixing and discharge must be shorter than the gel time for the mixed components or set time of the adhesive. Otherwise, gelling will occur with the nozzle, creating a clog. In the case of rapidly gelling bioadhesives, such as those designed for vascular sealing, gelling can occur almost immediately when the two component liquids are mixed. Existing applicator designs have significant empty space between the liquid feed tubes and the mixing/discharge features of the nozzle. This empty space creates opportunities for fluid recirculation and increases residence time of the fluids in the nozzle.

Existing airless atomized liquid spray nozzles and nozzle tips use a two channel swirl design and a two-piece insert/shell construction or a three-channel, offset-center design with a plastic shell and elastomeric tip. Uneven fluid feed channels cause problems in maintaining separation of a two-component fluid input upstream of the mixing cavity. In two-component spray applications, early mixing of the two components can cause premature gelling/solidification of the material, which can then clog the feed channels or discharge orifice. In a two or three channel design, which are both commonly available, both liquid components flow through each channel, introducing clogging potential. Moreover, these two and three channel designs require complex manufacturing methods.

### SUMMARY OF THE INVENTION

Embodiments of a delivery device, a kit comprising the delivery device, and methods of using the same are presented. In one embodiment, the delivery device includes a syringe assembly defining two or more reservoirs each configured to hold a dispensable material. Additionally, the delivery device may include a delivery tip. The delivery tip may have an inlet, an outlet, a central flow-restricting portion, and one or more channels adjacent to the central flow-restricting portion that provide fluid communication between the inlet and the outlet, the one or more channels including a section that tapers inwardly toward the outlet. In one embodiment where the delivery device is configured such that if the two or more reservoirs hold dispensable material then the syringe assembly can be actuated to dispense the dispensable material from the two or more reservoirs substantially simultaneously into the inlet.

In another embodiment, the delivery tip may have an inlet, a central flow-restricting portion, a distal portion comprising a nozzle that includes an outlet, and one or more channels adjacent to the central flow-restricting portion that provide fluid communication between the inlet and the outlet. In such an embodiment, the delivery tip may be characterized by a central axis running from the inlet to the outlet. The nozzle may include a section that tapers outwardly and away from the outlet along the central axis. In such an embodiment, the delivery device is configured such that if the two or more reservoirs hold dispensable material then the syringe assembly can be actuated to dispense the dispensable material from the two or more reservoirs substantially simultaneously into the inlet.

In one embodiment, the delivery tip includes a conduit portion having an inlet and a distal portion comprising a nozzle that includes an outlet. The delivery tip may also include a central flow-restricting portion disposed inside the conduit portion. The delivery tip may further comprise one or more channels adjacent to the central flow-restricting portion that provide fluid communication between the inlet and the outlet. In such an embodiment, the delivery tip may be characterized by a central axis running from the inlet to the outlet, and the nozzle including a section that tapers outwardly and away from the outlet along the central axis.

In another embodiment, the delivery tip may include one or more channels adjacent to the central flow-restricting portion that provide fluid communication between the inlet and the outlet. The one or more channels may include a section that tapers inwardly toward the outlet.

In a further embodiment of the delivery device, the one or more channels include multiple sections, each of which converges at an angle less than 90 degrees. Additionally, the one or more channels may include three sections, each of which converges at an angle less than 90 degrees. In one embodiment, the one or more channels include a swirl chamber inside the delivery tip and adjacent to the outlet. The delivery tip may characterized by a central axis running from the inlet to the outlet, and the swirl chamber may be configured such that it intersects a plane that is perpendicular to the central axis in a profile having three sides each defined by a line, the three lines forming a substantially equilateral triangle.

In one embodiment, the delivery tip may include a conduit, and the central flow-restricting portion may be coupled to but not integral with the conduit. The one or more channels may comprise at least some recesses in the central flow-restricting portion. In another embodiment, the one or more channels comprise at least some recesses in the conduit.

In one embodiment, the delivery device includes an attachment mechanism configured to couple the delivery tip to the syringe assembly. For example, the attachment mechanism may include one or more flanges that are integral with the delivery tip. In another embodiment, the attachment mechanism may include one or more threaded portions that are integral with the delivery tip.

A kit is also presented. In one embodiment, the kit includes an adhesive compound comprising a plurality of dispensable components. Additionally, the kit may include a syringe assembly defining two or more reservoirs each configured to hold one of the dispensable components. The kit may also include a delivery tip. The delivery tip may have an inlet, an outlet, a central flow-restricting portion, and one or more channels adjacent to the central flow-restricting portion that provide fluid communication between the inlet and the outlet. In one embodiment, the one or more channels include a section that tapers inwardly toward the outlet.

In one embodiment, the delivery tip may include an inlet, a central flow-restricting portion, a distal portion comprising a nozzle that includes an outlet, and one or more channels adjacent to the central flow-restricting portion that provide fluid communication between the inlet and the outlet. In such an embodiment, the delivery tip may be characterized by a central axis running from the inlet to the outlet. The nozzle may have a section that tapers outwardly and away from the outlet along the central axis.

Joinder references used in this disclosure (attached, coupled, connected) are to be construed broadly and may include intermediate members between a connection of elements and relative movement between elements. As such, joinder references do not necessarily infer that two elements are directly connected and in fixed relation to each other. In particular, the term “coupled” is defined as connected, although not necessarily directly, and not necessarily mechanically.

The terms “a” and “an” are defined as one or more unless this disclosure explicitly requires otherwise.

The term “substantially” and its variations are defined as being largely but not necessarily wholly what is specified as understood by one of ordinary skill in the art, and in one non-limiting embodiment “substantially” refers to ranges within 10%, preferably within 5%, more preferably within 1%, and most preferably within 0.5% of what is specified.

The terms “comprise” (and any form of comprise, such as “comprises” and “comprising”), “have” (and any form of have, such as “has” and “having”), “include” (and any form of include, such as “includes” and “including”) and “contain” (and any form of contain, such as “contains” and “containing”) are open-ended linking verbs. As a result, a method or device that “comprises,” “has,” “includes” or “contains” one or more steps or elements possesses those one or more steps or elements, but is not limited to possessing only those one or more elements. Likewise, a step of a method or an element of a device that “comprises,” “has,” “includes” or “contains” one or more features possesses those one or more features, but is not limited to possessing only those one or more features. Furthermore, a device or structure that is configured in a certain way is configured in at least that way, but may also be configured in ways that are not listed.

Other features and associated advantages will become apparent with reference to the following detailed description of specific embodiments in connection with the accompanying drawings.

### BRIEF DESCRIPTION OF THE DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers. The figures are drawn to scale (unless otherwise noted), meaning the sizes of the depicted elements are accurate relative to each other for at least the set of embodiments depicted in the figures.

FIG. 1 shows a perspective view of one embodiment of a delivery device;

FIG. 2 shows an exploded view of one embodiment of a delivery device;

FIG. 3 shows a side cross-section view of one embodiment of a delivery device;

FIG. 4 shows a perspective view of one embodiment of an applicator according to one embodiment of a delivery device;

FIG. 5 shows a rear view of one embodiment of an applicator according to one embodiment of a delivery device;

FIG. 6 shows a perspective exploded view of one embodiment of an applicator and delivery tip assembly;

FIG. 7 shows a perspective view of one embodiment of an assembled applicator and delivery tip;

FIG. 8 shows a side cross-section view of one embodiment of an assembled applicator and delivery tip;

FIG. 9 shows a perspective view of one embodiment of a conduit portion of a delivery tip;

FIG. 10 shows a side cross-section view of one embodiment of a conduit portion of a delivery tip;

FIG. 11 shows a perspective view of one embodiment of a conduit portion of a delivery tip;

FIG. 12 shows a perspective ghosted view of one embodiment of a delivery tip;

FIG. 13 shows a side ghosted view of another embodiment of a delivery tip;

FIG. 14 shows a distal ghosted view of the embodiment of a delivery tip described in accordance with the embodiment of FIG. 13;

FIG. 15 shows a distal ghosted view of the embodiment of a delivery tip described in accordance with the embodiment of FIG. 12;

FIG. 16 shows a perspective view of one embodiment of the central flow-restricting portion of the embodiment of the delivery tip described according to FIG. 12;

FIG. 17 shows a distal view of one embodiment of the central flow-restricting portion of the embodiment of the delivery tip described according to FIG. 12;

FIG. 18 shows a perspective view of one embodiment of the central flow-restricting portion of the embodiment of the delivery tip described according to FIG. 12;

FIG. 19 shows a perspective exploded view of another embodiment of an applicator and delivery tip;

FIG. 20 shows a perspective ghosted view of another embodiment of a delivery tip;

FIG. 21 shows a proximal perspective view of the embodiment of a delivery tip described in FIG. 20;

FIG. 22 shows a distal perspective view of another embodiment of a flow-restricting portion of a delivery tip;

FIG. 23 shows a proximal perspective view of the embodiment of a flow-restricting portion of the delivery tip described in FIG. 22;

FIG. 24 shows one embodiment of a kit comprising a delivery device according to one embodiment;

FIG. 25 shows a perspective view of one embodiment of an insertion tool for inserting a central flow-restricting portion of a delivery tip into a conduit portion of a delivery tip; and

FIG. 26 shows a side view of one embodiment of an insertion tool inserting a central flow-restricting portion of a delivery tip into a conduit portion of a delivery tip.

### DETAILED DESCRIPTION

Various features and advantageous details are explained more fully with reference to the nonlimiting embodiments that are illustrated in the accompanying drawings and detailed in the following description. Descriptions of well known starting materials, processing techniques, components, and equipment are omitted so as not to unnecessarily obscure the invention in detail. It should be understood, however, that the detailed description and the specific examples, while indicating embodiments of the invention, are given by way of illustration only, and not by way of limitation. Various substitutions, modifications, additions, and/or rearrangements within the spirit and/or scope of the underlying inventive concept as defined by the claims will become apparent to those skilled in the art from this disclosure.

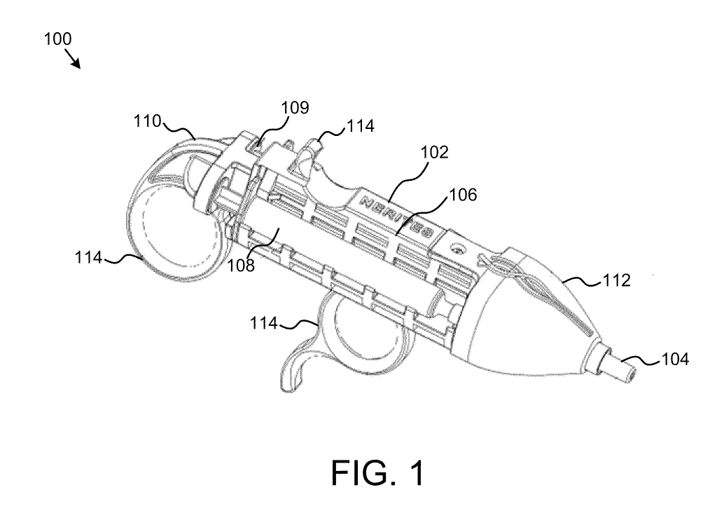

FIGS. 1-3 illustrate one embodiment of a delivery device 100. In the depicted embodiment, the delivery device 100 includes a syringe assembly 102 and a delivery tip 104. The delivery device 100 may also include an actuator 110 and an applicator housing 112.

In a general embodiment, the syringe assembly 102 may include a plurality of reservoirs. The reservoirs may each be configured to hold a dispensable material. For example, the reservoirs may include syringes 108 and 109. In certain embodiments, the syringe assembly 102 may also include a syringe holder 106. Alternatively, the syringe assembly may include a syringe housing (not shown). As shown in FIG. 1, the syringe holder 106 may provide mechanical support for the syringes 108 and 109. In a further embodiment, the syringe holder 106 may include a locking or attaching mechanism, such as a clamp, for securing the syringes 108 and 109 to the syringe holder 106.

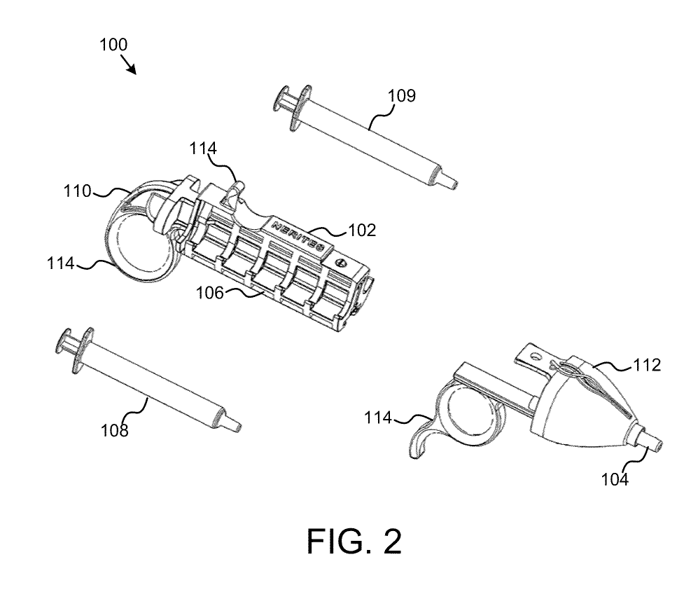

The actuator 110 may be coupled to the syringes 108 and 109. In one embodiment, if the syringes 108 and 109 hold dispensable material, then the syringe assembly 102 can be actuated by the actuator 110 to dispense the dispensable material from the syringes 108 and 109 substantially simultaneously. For example, if the syringe assembly 102 includes two syringes 108 and 109, as shown in FIGS. 1 and 2, the actuator 110 may cause both syringes 108 and 109 to dispense a dispensable material at substantially the same time. In a particular example, the dispensable material may be an adhesive compound having multiple dispensable components that, when mixed, form the adhesive. In a particular embodiment, a first syringe 108 may hold a first dispensable component and a second syringe 109 may hold a second dispensable component. The actuator 110 may cause both the first and the second dispensable components to be dispensed from the syringes 108 and 109 at substantially the same time.

To further facilitate dispensing of the dispensable material, and the like, the syringe assembly 102 may also include one or more handles 114. The handles 114 may include grips, loops, flanges, ribs, or the like. In certain embodiments, the handles 114 may be integrated with the actuator 110 to facilitate dispensing the dispensable material.

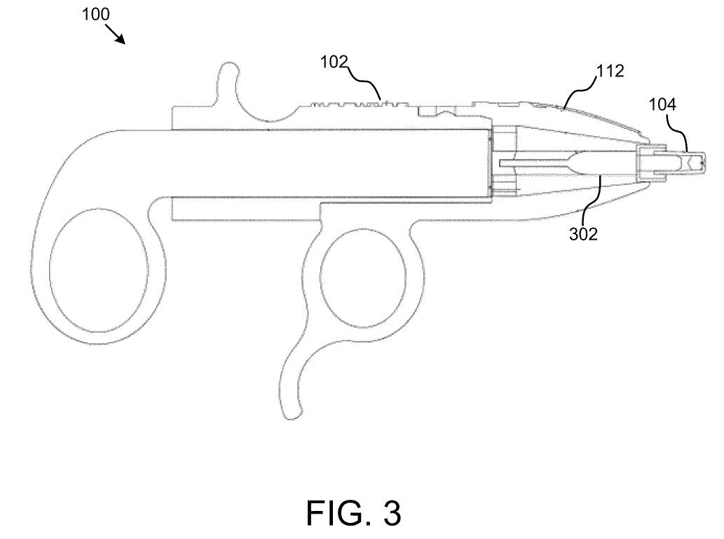

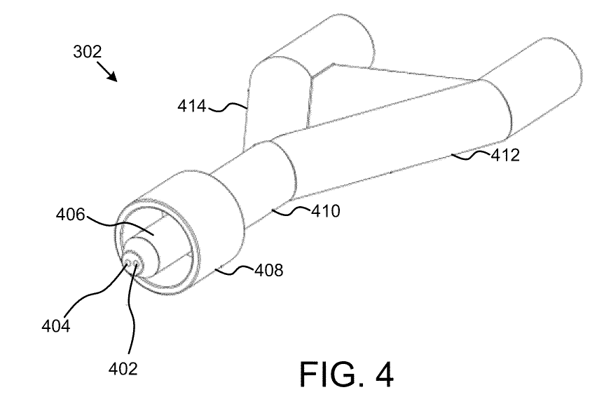

As shown in FIG. 3, the applicator housing 112 may house an applicator 302. The applicator 302 may couple the syringe assembly 102 to the delivery tip 104. In particular, the applicator 302 may provide fluid communication between the syringes 108 and 109 and the delivery tip 104. As illustrated in FIG. 4, the applicator 302 may include a first feed tube 410 having a first feed channel 402 and a second feed tube 414 having a second feed channel 404. The first feed channel 402 may couple a first syringe 108 to the delivery tip 104 and the second feed channel may couple a second syringe 109 to the delivery tip.

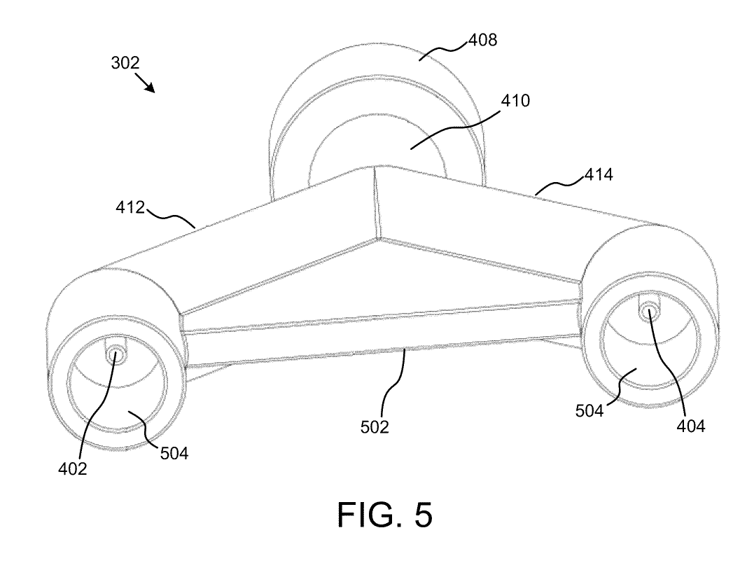

In a particular configuration as shown in FIG. 4, the applicator 302 may include a ‘Y’ shaped structure. In certain embodiments, the first feed channel 402 and the second feed channel 404 may converge to a singe channel within the body 410 of the applicator 302. Alternatively, the first feed channel 402 and the second feed channel may remain separated within the body 410 of the applicator 302. As illustrated in FIG. 5, the applicator 302 may include a plurality of inlets 504 each coupling the applicator 302 to a reservoir, such as a syringe 108 or 109. The applicator 302 may further comprise structural support, such as a bridge 502 supporting the first feed tube 412 and the second feed tube 414.

The applicator 302 may also include a first attachment member 408 configured for use in an attachment mechanism to a mating second attachment member 408. In one embodiment, the first attachment member 408 may include an annular collar. The annular collar may provide mechanical support for the deliver tip. In a further embodiment, the first attachment member 408 may include a portion of a locking device, such as a luer lock. In yet another embodiment, the first attachment member 408 may include a threaded collar configured to receive mating threads.

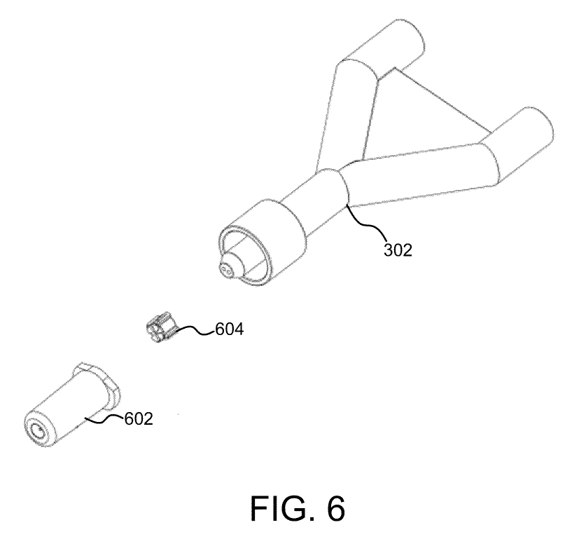

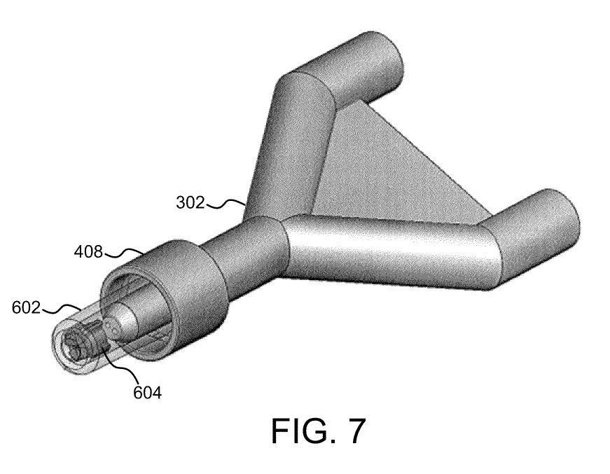

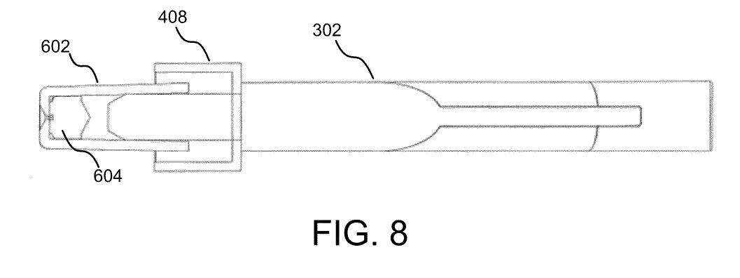

FIG. 6 illustrates one particular embodiment of an attachment mechanism. In the depicted embodiment, a conduit portion 602 of the delivery tip 104 and the applicator 302 include luer lock components for attaching the delivery tip 104 to the applicator 302. Additionally, FIG. 6 illustrates an exploded view of one embodiment of a delivery tip 104. As shown, the delivery tip 104 may include a conduit portion 602 and a central flow-restricting portion 604. The central flow-restricting portion 604 may be inserted into the conduit portion, and the conduit portion 602 may be attached to the applicator 302 or to the syringes 108 and 109 through alternative coupling means, such as tubes, couplers, or the like. FIGS. 7 and 8 illustrate one embodiment of the assembled conduit portion 602, central flow-restricting portion 604 and applicator 302.

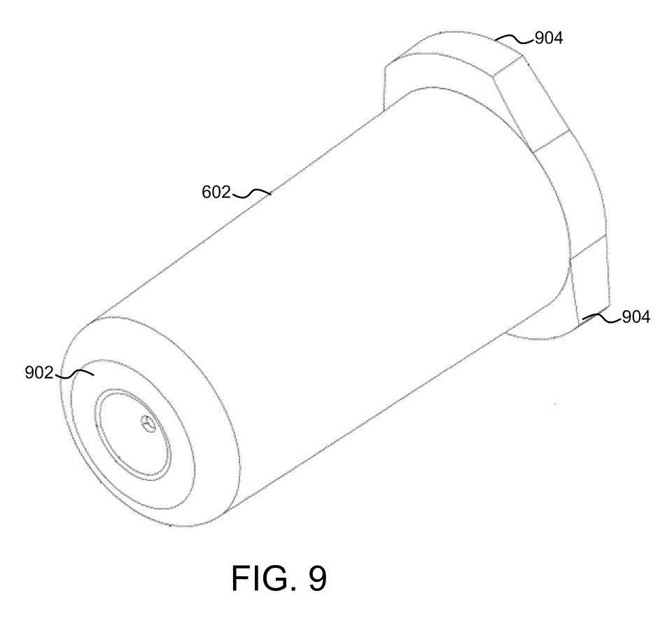

FIG. 9 illustrates a further embodiment of the conduit portion 602 of the delivery tip. In one embodiment, the conduit portion 602 includes a nozzle 902. The conduit portion 602 may also include one or more flanges 904 for mating with the attachment member 408. For example, the flanges 904 may mate with an annular collar of a luer lock assembly.

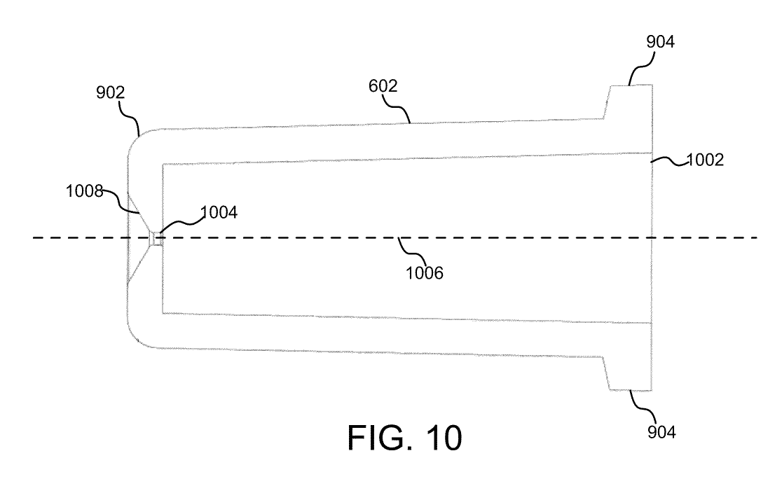

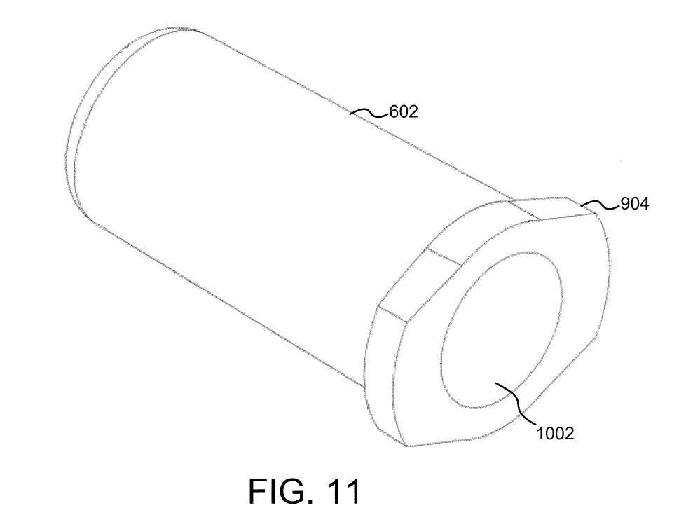

FIG. 10 describes the conduit portion 602 in greater detail. In one embodiment, the conduit portion 602 may include an inlet 1002 and a distal portion comprising a nozzle 902 that includes an outlet 1004. In such an embodiment, the delivery tip 104 may be characterized by a central axis 1006 running from the inlet 1002 to the outlet 1004. The nozzle 902 may include a section 1008 that tapers outwardly and away from the outlet 1004 along the central axis 1006. FIG. 11 further illustrates the inlet 1002 and the flange 904.

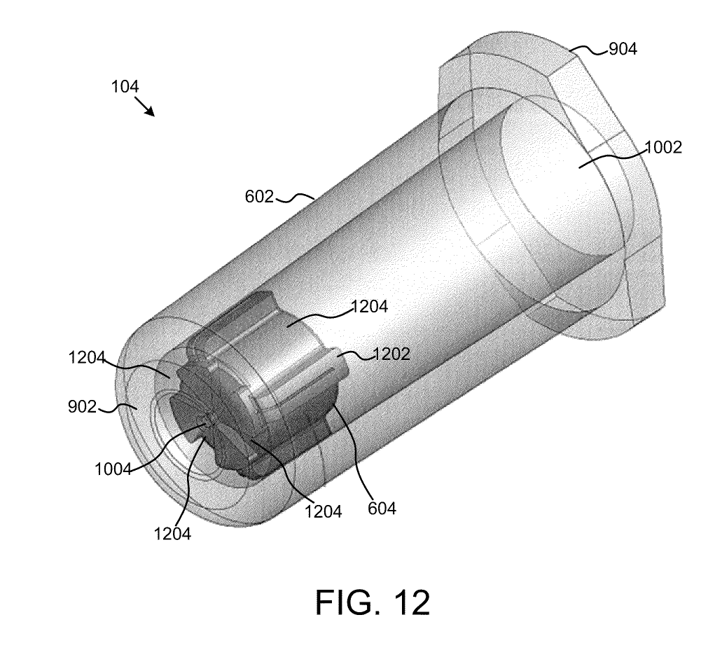

FIG. 12 shows one embodiment of an assembled delivery tip 104. The delivery tip 104 may include an inlet 1002, a central flow-restricting portion 604, a distal portion comprising a nozzle 902 that includes an outlet 1004, and one or more channels 1204 adjacent to the central flow-restricting portion 604 that provide fluid communication between the inlet 1002 and the outlet 1004. In such an embodiment, the delivery device is configured such that if the two or more reservoirs 108 and 109 hold dispensable material then the syringe assembly 102 can be actuated to dispense the dispensable material from the two or more reservoirs 108 and 109 substantially simultaneously into the inlet 1002.

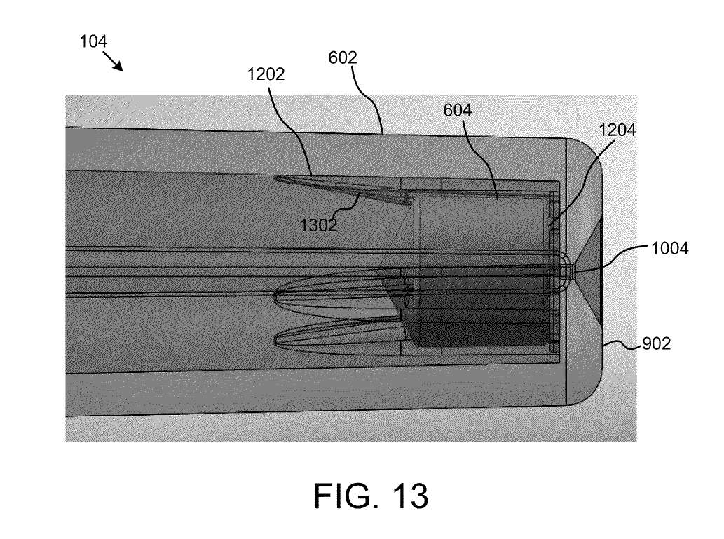

As illustrated in FIGS. 12 and 13, the delivery tip 104 may include a conduit portion 602 and a central flow-restricting portion 604. In one embodiment, the conduit portion 602 and the central flow-restricting portion 604 may be coupled but not integral. In an alternative embodiment, the central flow-restricting portion 604 may be integrally formed within the conduit portion 602. The one or more channels 1204 may comprise at least some recesses in the central flow-restricting portion 604. In another embodiment, the one or more channels 1204 comprise at least some recesses in the conduit 602. The recesses may be formed by ridges 1202 formed on the central flow-restricting portion 604 as shown in FIG. 12.

Alternatively, as shown in FIG. 13, the ridges 1202 may be formed on the conduit portion 602. In such an embodiment, the ridges 1202 may be further configured to retain the central flow-restricting portion 604 within the conduit portion 602. To facilitate insertion of the central flow-restricting portion 604, the ridges 1202 may include a tapered portion 1302 as illustrated. The tapered portion 1302 may additionally direct the dispensable material into the channels 1204. As shown in FIGS. 12 and 13, the channels 1204 may run adjacent to the central flow-restricting portion 604 and provide fluid communication between the inlet 1002 and the outlet 1004.

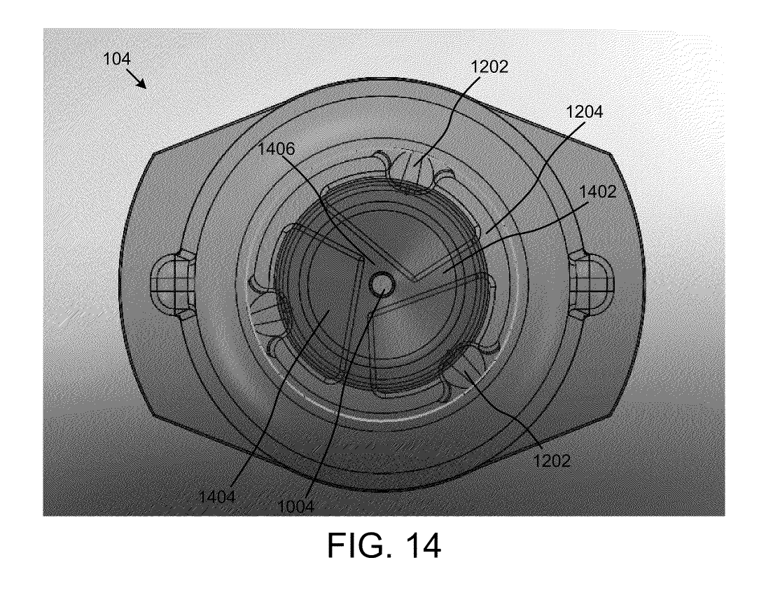

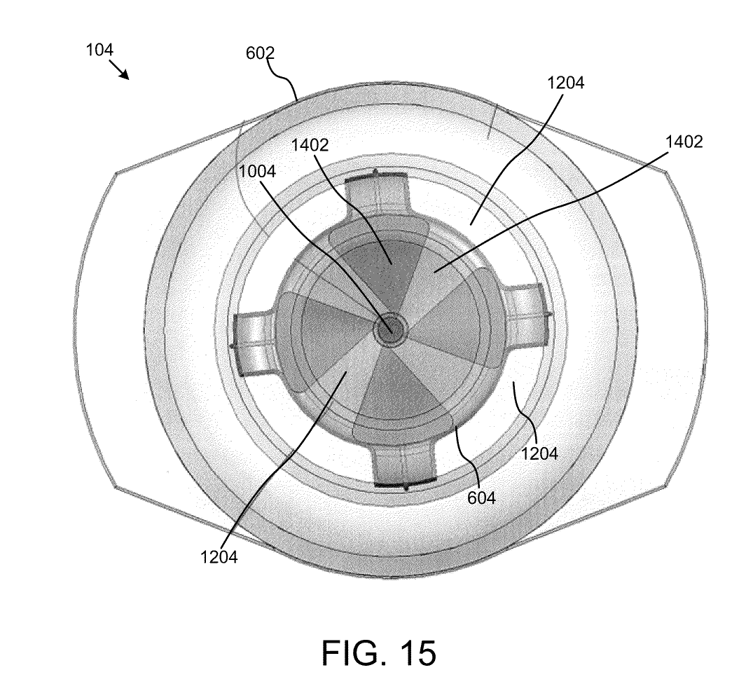

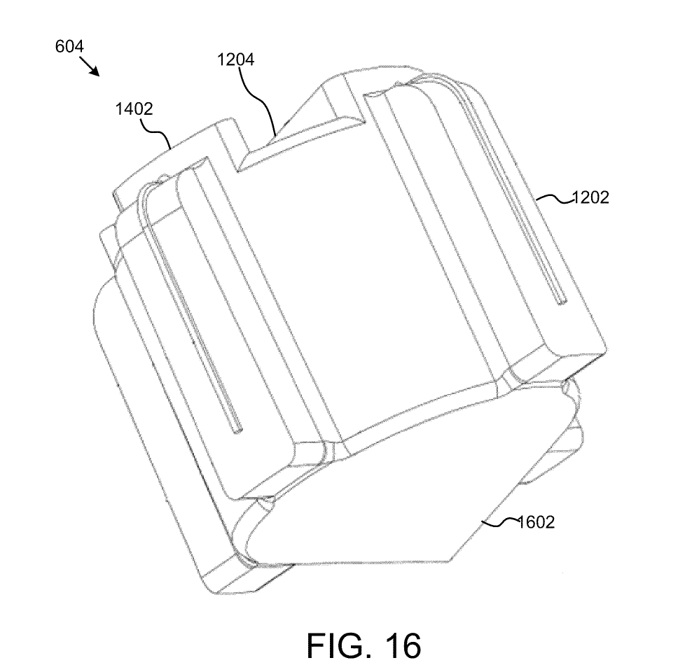

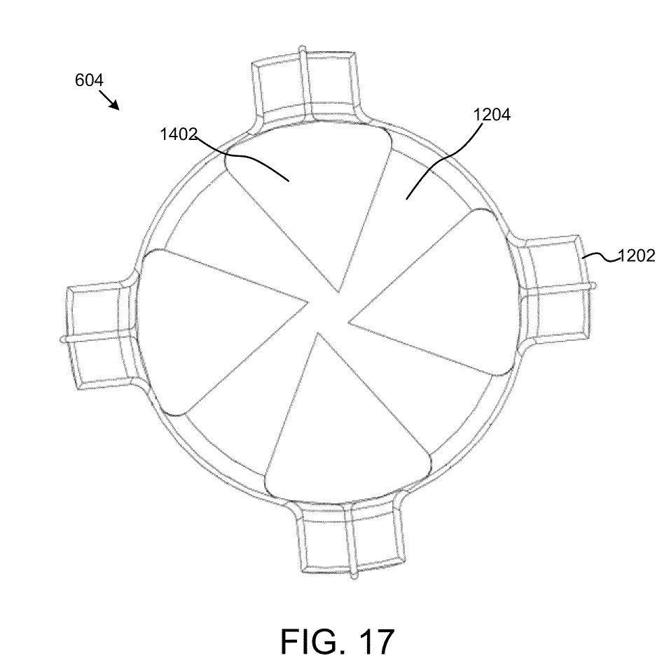

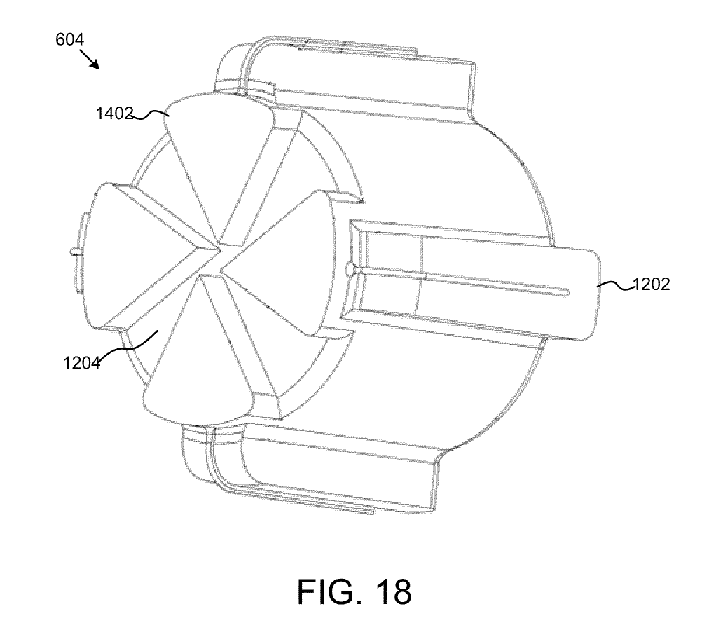

In a further embodiment, illustrated in FIG. 14, the one or more channels 1204 may include a section 1402 that tapers inwardly toward the outlet 1004. In a further embodiment, the one or more channels 1204 include multiple sections 1402, each of which converges at an angle less than 90 degrees. For example, as illustrated in FIG. 14, the one or more channels 1204 may include three sections 1402, each of which converges at an angle less than 90 degrees. In an alternative embodiment, illustrated in FIG. 15, the delivery tip 104 may include four channels 1204, each having a portion 1402 that converges at an angle less that 90 degrees. In one embodiment, the portions 1402 of the channels 1204 that converge may be formed between raised portions 1404 of the conduit portion 602. Alternatively, the raised portions 1404 may be formed on the central flow-restricting portion 604 as illustrated in FIGS. 15-18.

In one embodiment, the one or more channels 1204 include a swirl chamber 1406 inside the delivery tip 104 and adjacent to the outlet 1004. The delivery tip 104 may be characterized by a central axis 1006 running from the inlet 1002 to the outlet 1004, and the swirl chamber 1406 may be configured such that it intersects a plane that is perpendicular to the central axis 1006 in a profile having three sides each defined by a line, the three lines forming a substantially equilateral triangle as illustrated in FIG. 14.

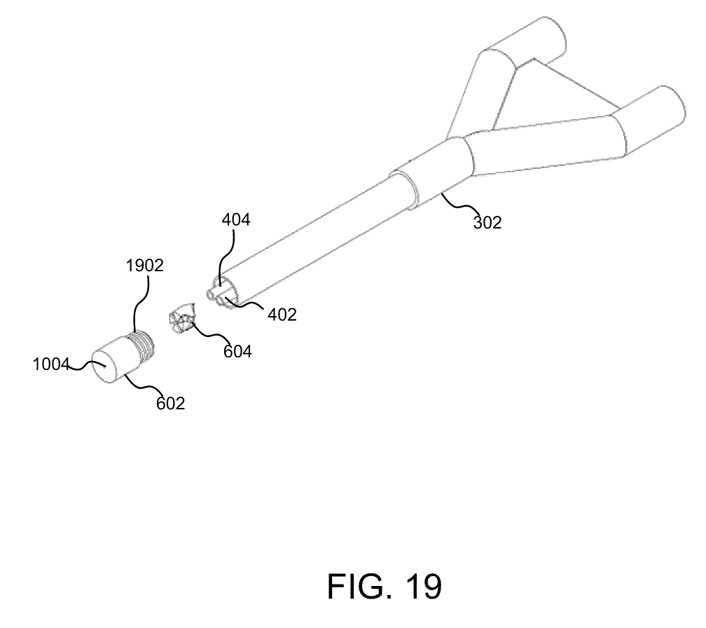

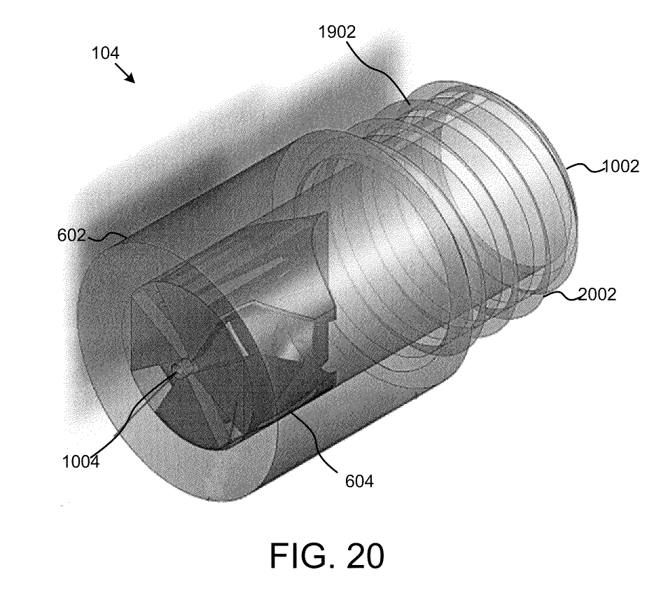

FIG. 19 illustrates a further embodiment of an assembly of the conduit 602, the central flow-restricting portion 604, and the applicator 302. In the described embodiment, the first feed channel 402 and the second feed channel 404 may include hoses or pipes running through the body of the applicator 302. Additionally, the attachment mechanism may include a threaded portion 1902 of the conduit portion 602 and a mating threaded portion (not shown) on the applicator 302. The threaded portion 1902 may include one or more threads 2002 as illustrated in FIG. 20.

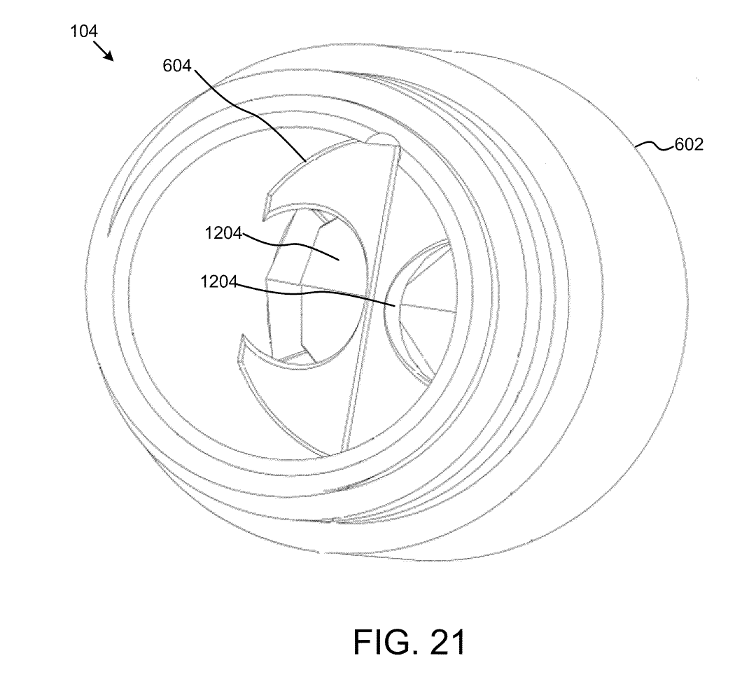

In this embodiment, the central flow-restricting portion 602 may also be inserted into the conduit portion 602 as shown in FIG. 20. Various configurations of the channels 1204 running adjacent to the central flow-restricting portion 604 may be formed, depending upon the geometry of the central flow-restricting portion 604 and the conduit portion 602. For example, in FIG. 21, the channels 1204 may have a substantially circular cross-section. Alternatively, the channels may have a substantially rectangular, square, oval, or other geometrically shaped cross-section.

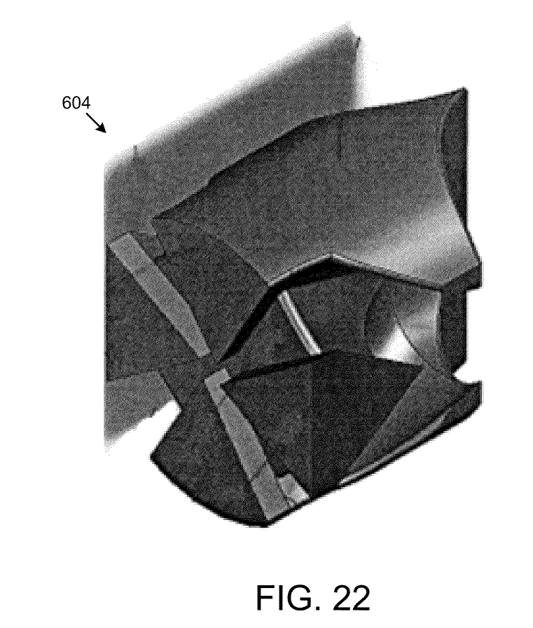

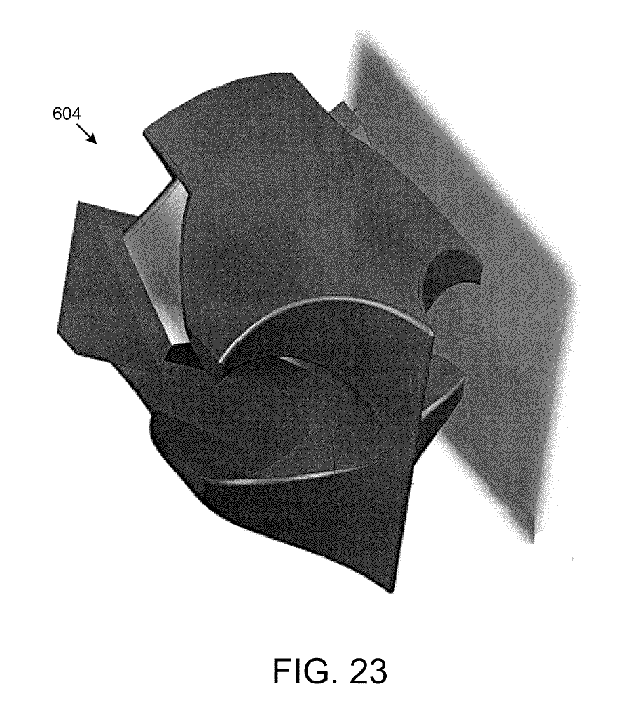

Indeed, as illustrated in FIGS. 22 and 23, the geometry of the central flow-restricting portion 604 may be selected to enhance certain flow characteristics, including flow-rate, clog susceptibility, fluid rotation, swirl velocity, and the like. For example, the channel may converge as it nears the outlet 1004.

The viscosity of the dispensable material may affect the spray performance. In one embodiment, the viscosity of the dispensable may be within a predetermined range of the viscosity of water at room-temperature (˜1 cP).

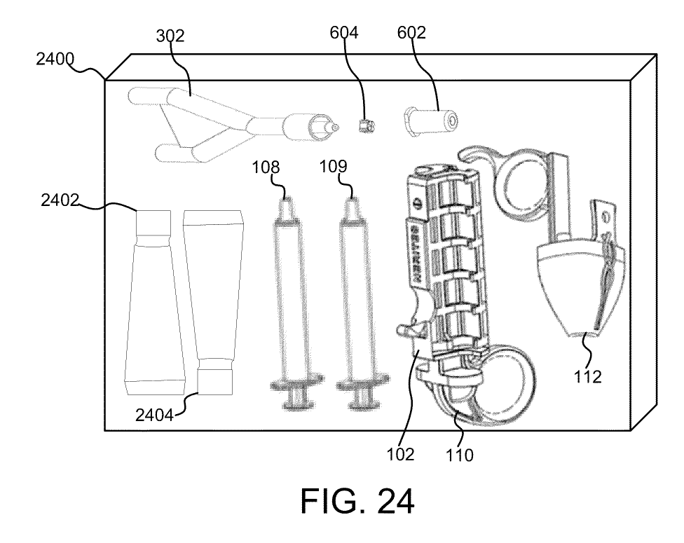

FIG. 24 shows one embodiment of a kit 2400 comprising a delivery device 100 according to one embodiment. The kit 2400 may include an adhesive compound comprising a plurality of dispensable components. The dispensable components may be stored in separate containers 2402, 2404. For example, a first container 2402 may hold a first dispensable component, and a second container 2404 may hold a second dispensable component. In one embodiment, the containers 2402, 2404 may hold the dispensable components during shipment. In such an embodiment, the two or more reservoirs 108 and 109 may be filled with the dispensable components from the containers 2402, 2404. Alternatively, the containers 2402, 2404 may be eliminated, and the dispensable components may be shipped directly in the reservoirs 108 and 109.

The kit 2400 may include a syringe assembly 102, and an applicator housing 112. The kit 2400 may also include an applicator 302. In a further embodiment, the kit may include a delivery tip 104, comprising a conduit portion 602 and a central flow-restricting portion 604. The kit 2400 may be shipped in a housing, box, or other suitable packaging. In certain embodiments, the components and packaging of the kit 2400 may be sterilized and hermetically sealed to avoid contamination. Thus, the kit 2400 may be ready for use during surgery without the need for additional sterilization of the components of the kit 2400.

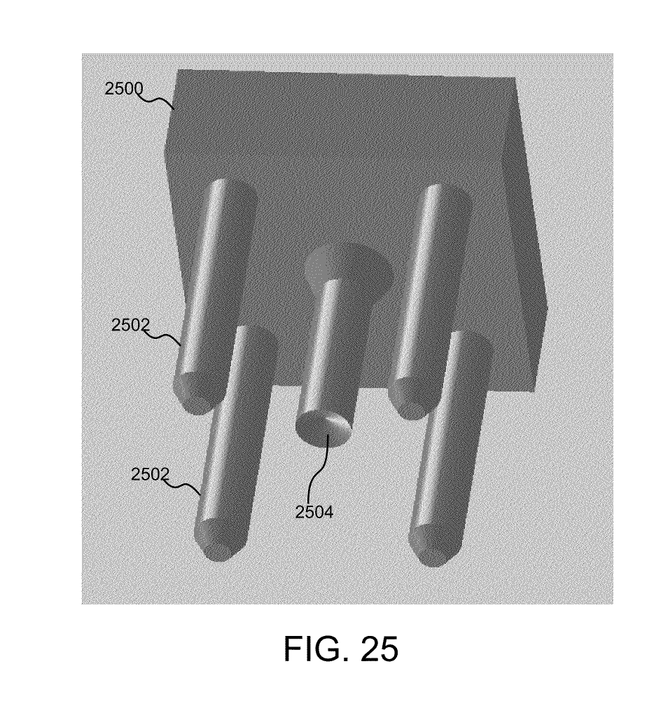

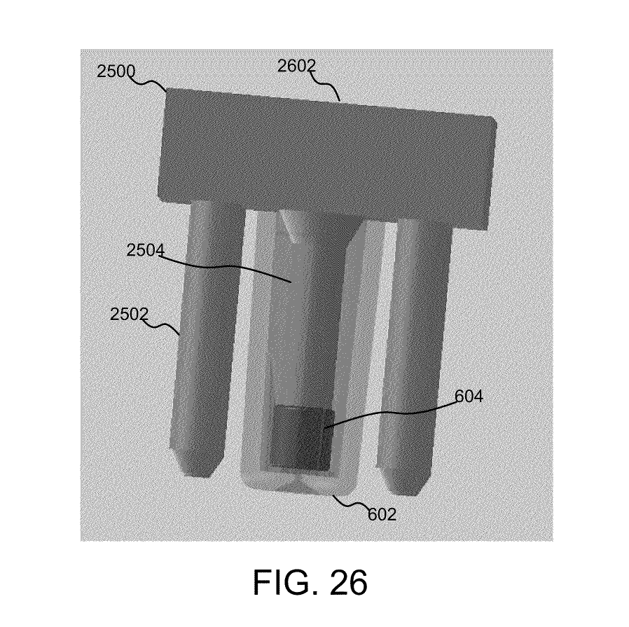

FIGS. 25-26 illustrate one embodiment of an insertion tool 2500 for inserting the central flow-restricting portion 604 into the conduit portion 602 of the delivery tip 104. In one embodiment, the insertion tool 2500 may include a ram-rod 2504. In a further embodiment, the insertion tool 2500 may include one or more alignment posts 2502 for aligning the insertion tool in a press (not shown). As shown in FIG. 26, the central flow-flow restricting portion 604 may be inserted into the conduit portion 602. The ram-rod 2504 may also be inserted into the conduit portion 602. In a further embodiment, pressure may be applied to a top surface 2602 of the insertion tool 2500 until the central flow-restricting portion 604 has been inserted into the conduit portion 604 to within a predetermined range of a predetermined depth.

In some embodiments, the delivery device 100 described herein consists of components for preparation of a synthetic polymeric adhesive/sealant, and an applicator for delivery of the sealant to a target site, such as a surgical site or wound. The sealant comprises two solutions: a polyethylene glycol (PEG) solution and a cross-linking solution. When mixed together, the precursors rapidly cross-link to form the hydrogel sealant. The mixing of the precursors is accomplished as the materials exit the delivery tip 104 of the delivery device 100.

In one embodiment, a kit is provided that includes the delivery device 100, including syringes 108 and 109, adhesive components and instructions for the application of the adhesive material. Kits may further comprise any other device, material, or component useful for any medical procedure in which the delivery device 100 is employed. Such additional devices, materials, and components include, but are not limited to, drugs (e.g., anesthetics, coagulants, vasoconstrictors), meshes, sutures, dressings, cutting devices, hemostats, clamps, disinfectants, sponges, soaps, retractors, gloves, masks, gowns, syringes, needles, and the like.

Suitable polymeric materials useful to prepare the adhesive material include, but are not limited to, those composed of dihydroxyphenylalanine derivatives (DHP), such as 3,4-dihydroxyphenylalanine (DOPA), dopamine, 3,4-dihydroxyhydroccinamic acid (DOHA), and other catechols, and polymers of known biocompatibilities (e.g., polyethylene glycol (PEG)) and biodegradability (e.g., polycaprolactone (PCL), polylactic acid (PLA), polyglycolic acid (PGA), or various combination of the polyesters). Additionally, other DHP-modified polymers can be used. Non-limiting examples of such polymers include those described in US Pub. No. 20080247984, US Pub. No. 20030087338, US Pub. No. 20050288398, US Pub. No. 20060009550, US Pub. No. 20060241281, US Pub. No. 20070208141, US Pub. No. 20080149566, US Pub. No. 20080169059, US Pub. No. 20080171012, US Pub. No. 20080247984, US Pub. No. 20090123652, and US Pub. No. 20090163661, and U.S. Appln. Ser. Nos. 60/821,459, 11/834,651, 12/239,787, 61/100,742, 61/150,471, 61/100,781, 61/100,560, 61/100,738, 61/150,483, 61/160,479, and 61/150,464, each of which is herein incorporated by reference in its entirety.

The invention further provides crosslinked hydrogels derived from the compositions described herein. For example, two dihydroxyphenyl (DHPD) moieties from two separate polymer chains can be reacted to form a bond between the two DHPD moieties, e.g., catechols. Typically, this is an oxidative/radical initiated crosslinking reaction wherein oxidants/initiators such as periodates and iodates, such as NaIO₄ or KIO₄, NaIO₃ or KIO₃ and the like, FeCl₃, H₂O₂, oxygen, an inorganic base, an organic base or an enzymatic oxidase can be used. Typically, a ratio of oxidant/initiator to DHPD containing material is between about 0.2 to about 1.0 (on a molar basis) (oxidant:DHPD). In one particular embodiment, the ratio is between about 0.25 to about 0.75 and more particularly between about 0.4 to about 0.6 (e.g., 0.5). It has been found that periodate is very effective in the preparation of crosslinked hydrogels described herein.

A typical procedure to prepare the adhesive coating is described as follows.

1. Open a transfer valve between the diluent syringe 108 and the powder syringe containing polymer (not shown) using an attached luer lock transfer valve.

2. Transfer the diluent into polymer powder by gently depressing the plunger on the diluent syringe 108. Mix contents back and forth between the syringes at 20-30 times, or until the solid is completely dissolved. Push contents into the diluent syringe 108. Discard powder syringe and transfer valve.

3. Expel air from the diluent syringe 108.

4. It is preferred that the diluent syringe 108 and crosslinker syringe 109 contain equal volumes of liquid. If fluid levels are not equal, expel fluids out of syringes until equal.

5. Attach the two precursor syringes 108 and 109 to the “Y”-shaped applicator 302.

6. While holding syringes 108 and 109 by the barrels, seat the syringes into the syringe assembly and attach a plunger link to the plungers of both syringes 108 and 109 without dispensing precursors into the applicator.

7. Attach one delivery tip 104 to the end of the applicator.

8. Position the delivery device 100 approximately 4 cm above the target site. Apply even pressure to the center of the plunger link to dispense the precursors. To achieve best results begin application by spraying on gauze. Move applicator directly to the target site in a single motion.

9. Continue applying the hydrogel until a thin (approx. 1-2 mm) coating is formed. Maintain 2-3 mm margins around a suture line. Gelation will occur within a few seconds.

10. Excess hydrogel may be removed with scissors or mechanical disruption. Irrigation immediately after the sealant has solidified is permitted.

The following paragraphs enumerated consecutively from 1 through 63 provide for various aspects of the present invention. In one embodiment, in a first paragraph (1), the present invention provides a delivery device comprising:

a syringe assembly defining two or more reservoirs each configured to hold a dispensable material; and

a delivery tip having an inlet, an outlet, a central flow-restricting portion, and one or more channels adjacent the central flow-restricting portion that provide fluid communication between the inlet and the outlet, the one or more channels including a section that tapers inwardly toward the outlet;

where the delivery device is configured such that if the two or more reservoirs hold dispensable material then the syringe assembly can be actuated to dispense the dispensable material from the two or more reservoirs substantially simultaneously into the inlet.

2. The delivery device of paragraph 1, where the one or more channels include multiple sections, each of which converges at an angle less than 90 degrees.

3. The delivery device of paragraph 2, where the one or more channels include three sections, each of which converges at an angle less than 90 degrees.

4. The delivery device of paragraph 1, where the one or more channels include a swirl chamber inside the delivery tip and adjacent to the outlet.

5. The delivery device of paragraph 4, where the delivery tip is characterized by a central axis running from the inlet to the outlet, and the swirl chamber is configured such that it intersects a plane that is perpendicular to the central axis in a profile having three sides each defined by a line, the three lines forming a substantially equilateral triangle.

6. The delivery device of paragraph 1, where the delivery tip includes a conduit, and the central flow-restricting portion is coupled to but not integral with the conduit.

7. The delivery device of paragraph 6, where the one or more channels comprise at least some recesses in the central flow-restricting portion.

8. The delivery device of paragraph 6, where the one or more channels comprise at least some recesses in the conduit.

9. The delivery device of paragraph 1, further comprising an attachment mechanism configured to couple the delivery tip to the syringe assembly.

10. The delivery device of paragraph 9, where the attachment mechanism comprises one or more flanges that are integral with the delivery tip.

11. A delivery device comprising:

a syringe assembly defining two or more reservoirs each configured to hold a dispensable material; and

a delivery tip having an inlet, a central flow-restricting portion, a distal portion comprising a nozzle that includes an outlet, and one or more channels adjacent the central flow-restricting portion that provide fluid communication between the inlet and the outlet, the delivery tip being characterized by a central axis running from the inlet to the outlet, the nozzle including a section that tapers outwardly and away from the outlet along the central axis;

where the delivery device is configured such that if the two or more reservoirs hold dispensable material then the syringe assembly can be actuated to dispense the dispensable material from the two or more reservoirs substantially simultaneously into the inlet.

12. The delivery device of paragraph 11, where the one or more channels include multiple sections, each of which converges at an angle less than 90 degrees.

13. The delivery device of paragraph 12, where the one or more channels include three sections, each of which converges at an angle less than 90 degrees.

14. The delivery device of paragraph 11, where the one or more channels include a swirl chamber inside the delivery tip and adjacent to the outlet.

15. The delivery device of paragraph 14, where the delivery tip is characterized by a central axis from the inlet to the outlet, and the swirl chamber is configured such that it intersects a plane that is perpendicular to the central axis in a profile having three sides each defined by a line, the three lines forming a substantially equilateral triangle.

16. The delivery device of paragraph 11, where the delivery tip includes a conduit, and the central flow-restricting portion is coupled to but not integral with the conduit.

17. The delivery device of paragraph 16, where the one or more channels comprise at least some recesses in the central flow-restricting portion.

18. The delivery device of paragraph 16, where the one or more channels comprise at least some recesses in the conduit.

19. The delivery device of paragraph 11, further comprising an attachment mechanism configured to couple the delivery tip to the syringe assembly.

20. The delivery device of paragraph 19, where the attachment mechanism comprises one or more flanges that are integral with the delivery tip.

21. A delivery tip comprising:

a conduit portion having an inlet and a distal portion comprising a nozzle that includes an outlet;

a central flow-restricting portion disposed inside the conduit portion; and

one or more channels adjacent the central flow-restricting portion that provide fluid communication between the inlet and the outlet;

where the delivery tip is characterized by a central axis running from the inlet to the outlet, and the nozzle including a section that tapers outwardly and away from the outlet along the central axis.

22. The delivery tip of paragraph 21, where the central flow-restricting portion is coupled to but not integral with the conduit portion.

23. The delivery tip of paragraph 21, where the one or more channels include multiple sections, each of which converges at an angle less than 90 degrees.

24. The delivery tip of paragraph 23, where the one or more channels include three sections, each of which converges at an angle less than 90 degrees.

25. The delivery tip of paragraph 21, where the one or more channels include a swirl chamber inside the delivery tip and adjacent to the outlet.

26. The delivery tip of paragraph 25, where the delivery tip is characterized by a central axis from the inlet to the outlet, and the swirl chamber is configured such that it intersects a plane that is perpendicular to the central axis in a profile having three sides each defined by a line, the three lines forming a substantially equilateral triangle.

27. The delivery tip of paragraph 21, where the delivery tip includes a conduit, and the central flow-restricting portion is coupled to but not integral with the conduit.

28. The delivery tip of paragraph 27, where the one or more channels comprise at least some recesses in the central flow-restricting portion.

29. The delivery tip of paragraph 27, where the one or more channels comprise at least some recesses in the conduit.

30. The delivery tip of paragraph 21, further comprising an attachment mechanism configured to couple the delivery tip to the syringe assembly.

31. The delivery tip of paragraph 30, where the attachment mechanism comprises one or more flanges that are integral with the delivery tip.

32. A delivery tip comprising:

a conduit portion having an inlet and a distal portion comprising a nozzle that includes an outlet;

a central flow-restricting portion disposed inside the conduit portion; and

one or more channels adjacent the central flow-restricting portion that provide fluid communication between the inlet and the outlet, the one or more channels including a section that tapers inwardly toward the outlet.

33. The delivery tip of paragraph 32, where the central flow-restricting portion is coupled to but not integral with the conduit portion.

34. The delivery tip of paragraph 32, where the one or more channels include multiple sections, each of which converges at an angle less than 90 degrees.

35. The delivery tip of paragraph 34, where the one or more channels include three sections, each of which converges at an angle less than 90 degrees.

36. The delivery tip of paragraph 32, where the one or more channels include a swirl chamber inside the delivery tip and adjacent to the outlet.

37. The delivery tip of paragraph 36, where the delivery tip is characterized by a central axis from the inlet to the outlet, and the swirl chamber is configured such that it intersects a plane that is perpendicular to the central axis in a profile having three sides each defined by a line, the three lines forming a substantially equilateral triangle.

38. The delivery tip of paragraph 32, where the delivery tip includes a conduit, and the central flow-restricting portion is coupled to but not integral with the conduit.

39. The delivery tip of paragraph 38, where the one or more channels comprise at least some recesses in the central flow-restricting portion.

40. The delivery tip of paragraph 38, where the one or more channels comprise at least some recesses in the conduit.

41. The delivery tip of paragraph 32, further comprising an attachment mechanism configured to couple the delivery tip to the syringe assembly.

42. The delivery tip of paragraph 41, where the attachment mechanism comprises one or more flanges that are integral with the delivery tip.

43. A kit comprising:

an adhesive compound comprising a plurality of dispensable components;

a syringe assembly defining two or more reservoirs each configured to hold one of the dispensable components; and

a delivery tip having an inlet, an outlet, a central flow-restricting portion, and one or more channels adjacent the central flow-restricting portion that provide fluid communication between the inlet and the outlet, the one or more channels including a section that tapers inwardly toward the outlet;

where the delivery device is configured such that if the two or more reservoirs hold dispensable components then the syringe assembly can be actuated to dispense the dispensable components from the two or more reservoirs substantially simultaneously into the inlet.

44. The kit of paragraph 43, where the one or more channels include multiple sections, each of which converges at an angle less than 90 degrees.

45. The kit of paragraph 44, where the one or more channels include three sections, each of which converges at an angle less than 90 degrees.

46. The kit of paragraph 43, where the one or more channels include a swirl chamber inside the delivery tip and adjacent to the outlet.

47. The kit of paragraph 46, where the delivery tip is characterized by a central axis running from the inlet to the outlet, and the swirl chamber is configured such that it intersects a plane that is perpendicular to the central axis in a profile having three sides each defined by a line, the three lines forming a substantially equilateral triangle.

48. The kit of paragraph 43, where the delivery tip includes a conduit, and the central flow-restricting portion is coupled to but not integral with the conduit.

49. The kit of paragraph 48, where the one or more channels comprise at least some recesses in the central flow-restricting portion.

50. The kit of paragraph 48, where the one or more channels comprise at least some recesses in the conduit.

51. The kit of paragraph 43, further comprising an attachment mechanism configured to couple the delivery tip to the syringe assembly.

52. The kit of paragraph 51, where the attachment mechanism comprises one or more flanges that are integral with the delivery tip.

53. A kit comprising:

an adhesive compound comprising a plurality of dispensable components;

a syringe assembly defining two or more reservoirs each configured to hold a dispensable component; and

a delivery tip having an inlet, a central flow-restricting portion, a distal portion comprising a nozzle that includes an outlet, and one or more channels adjacent the central flow-restricting portion that provide fluid communication between the inlet and the outlet, the delivery tip being characterized by a central axis running from the inlet to the outlet, the nozzle including a section that tapers outwardly and away from the outlet along the central axis;

where the delivery device is configured such that if the two or more reservoirs hold dispensable component then the syringe assembly can be actuated to dispense the dispensable component from the two or more reservoirs substantially simultaneously into the inlet.

54. The kit of paragraph 53, where the one or more channels include multiple sections, each of which converges at an angle less than 90 degrees.

55. The kit of paragraph 54, where the one or more channels include three sections, each of which converges at an angle less than 90 degrees.

56. The kit of paragraph 53, where the one or more channels include a swirl chamber inside the delivery tip and adjacent to the outlet.

57. The kit of paragraph 56, where the delivery tip is characterized by a central axis running from the inlet to the outlet, and the swirl chamber is configured such that it intersects a plane that is perpendicular to the central axis in a profile having three sides each defined by a line, the three lines forming a substantially equilateral triangle.

58. The kit of paragraph 53, where the delivery tip includes a conduit, and the central flow-restricting portion is coupled to but not integral with the conduit.

59. The kit of paragraph 58, where the one or more channels comprise at least some recesses in the central flow-restricting portion.

60. The kit of paragraph 58, where the one or more channels comprise at least some recesses in the conduit.

61. The kit of paragraph 53, further comprising an attachment mechanism configured to couple the delivery tip to the syringe assembly.

62. The kit of paragraph 61, where the attachment mechanism comprises one or more flanges that are integral with the delivery tip.

63. A method to seal a surgical site or a wound comprising the step of applying an adhesive that is admixed in a delivery device comprising:

a syringe assembly defining a first reservoir and a second reservoir, each configured to hold a dispensable material, wherein the first reservoir contains a solution of a polymeric catechol material and the second reservoir contains a crosslinking solution of a periodate; and

a delivery tip having an inlet, an outlet, a central flow-restricting portion, and one or more channels adjacent the central flow-restricting portion that provide fluid communication between the inlet and the outlet, the one or more channels including a section that tapers inwardly toward the outlet;

where the delivery device is configured such that if the two or more reservoirs hold dispensable material then the syringe assembly can be actuated to dispense the dispensable material from the two or more reservoirs substantially simultaneously into the inlet.

It is intended that all matter contained in the above description or shown in the accompanying drawings shall be interpreted as illustrative only and not limiting. Although various representative embodiments of this invention have been described above with a certain degree of particularity, those skilled in the art having the benefit of this disclosure will recognize that numerous alterations could be made to the disclosed embodiments without departing from the spirit or scope of the inventive subject matter set forth in the claims. For example, in methodologies directly or indirectly set forth herein, various steps and operations are described in one possible order of operation, but those skilled in the art will recognize that steps and operations may be rearranged, replaced, or eliminated and still fall within the scope of the claims. For example, as described in the figures above, various embodiments of a central flow-restricting portion 604 may be used in conjunction with various embodiments of a conduit portion 602. In particular, the shape and configuration of the channels may be altered to modify the flow characteristics of the delivery tip 104. For example, the present embodiments have been described with three or four channels 1204, but more or fewer channels may be used. In particular, the delivery tip 104 may include only two channels 1204. Alternatively, an embodiment of the delivery tip 104 may include five channels 1204.

The claims are not to be interpreted as including means-plus- or step-plus-function limitations, unless such a limitation is explicitly recited in a given claim using the phrase(s) “means for” or “step for,” respectively.

What is claimed is:

1. A delivery device comprising:
a syringe assembly defining two or more reservoirs each configured to hold a dispensable material; and
a delivery tip having an inlet, an outlet, a central flow-restricting portion, and one or more channels adjacent the central flow-restricting portion that provide fluid communication between the inlet and the outlet, the one or more channels including a section that tapers inwardly toward the outlet;
where the delivery device is configured such that if the two or more reservoirs hold dispensable material then the syringe assembly can be actuated to dispense the dispensable material from the two or more reservoirs substantially simultaneously into the inlet.

2. The delivery device of claim 1, where the one or more channels include multiple sections, each of which converges at an angle less than 90 degrees.

3. The delivery device of claim 2, where the one or more channels include three sections, each of which converges at an angle less than 90 degrees.

4. The delivery device of claim 1, where the one or more channels include a swirl chamber inside the delivery tip and adjacent to the outlet.

5. The delivery device of claim 4, where the delivery tip is characterized by a central axis running from the inlet to the outlet, and the swirl chamber is configured such that it intersects a plane that is perpendicular to the central axis in a profile having three sides each defined by a line, the three lines forming a substantially equilateral triangle.

6. The delivery device of claim 1, where the delivery tip includes a conduit, and the central flow-restricting portion is coupled to but not integral with the conduit.

7. The delivery device of claim 6, where the one or more channels comprise at least some recesses in the central flow-restricting portion.

8. The delivery device of claim 6, where the one or more channels comprise at least some recesses in the conduit.

9. The delivery device of claim 1, further comprising an attachment mechanism configured to couple the delivery tip to the syringe assembly.

10. The delivery device of claim 9, where the attachment mechanism comprises one or more flanges that are integral with the delivery tip.

11. A delivery device comprising:
a syringe assembly defining two or more reservoirs each configured to hold a dispensable material; and
a delivery tip having an inlet, a central flow-restricting portion, a distal portion comprising a nozzle that includes an outlet, and one or more channels adjacent the central flow-restricting portion that provide fluid communication between the inlet and the outlet, the delivery tip being characterized by a central axis running from the inlet to the outlet, the nozzle including a section that tapers outwardly and away from the outlet along the central axis;
where the delivery device is configured such that if the two or more reservoirs hold dispensable material then the syringe assembly can be actuated to dispense the dispensable material from the two or more reservoirs substantially simultaneously into the inlet.

12. The delivery device of claim 11, where the one or more channels include multiple sections, each of which converges at an angle less than 90 degrees.

13. The delivery device of claim 12, where the one or more channels include three sections, each of which converges at an angle less than 90 degrees.

14. The delivery device of claim 11, where the one or more channels include a swirl chamber inside the delivery tip and adjacent to the outlet.

15. The delivery device of claim 14, where the delivery tip is characterized by a central axis from the inlet to the outlet, and the swirl chamber is configured such that it intersects a plane that is perpendicular to the central axis in a profile having three sides each defined by a line, the three lines forming a substantially equilateral triangle.

16. The delivery device of claim 11, where the delivery tip includes a conduit, and the central flow-restricting portion is coupled to but not integral with the conduit.

17. The delivery device of claim 16, where the one or more channels comprise at least some recesses in the central flow-restricting portion.

18. The delivery device of claim 16, where the one or more channels comprise at least some recesses in the conduit.

19. The delivery device of claim 11, further comprising an attachment mechanism configured to couple the delivery tip to the syringe assembly.

20. The delivery device of claim 19, where the attachment mechanism comprises one or more flanges that are integral with the delivery tip.

21. A delivery tip comprising:
a conduit portion having an inlet and a distal portion comprising a nozzle that includes an outlet;
a central flow-restricting portion disposed inside the conduit portion; and
one or more channels adjacent the central flow-restricting portion that provide fluid communication between the inlet and the outlet;
where the delivery tip is characterized by a central axis running from the inlet to the outlet, and the nozzle including a section that tapers outwardly and away from the outlet along the central axis.

22. The delivery tip of claim 21, where the central flow-restricting portion is coupled to but not integral with the conduit portion.

23. The delivery tip of claim 21, where the one or more channels include multiple sections, each of which converges at an angle less than 90 degrees.

24. The delivery tip of claim 23, where the one or more channels include three sections, each of which converges at an angle less than 90 degrees.

25. The delivery tip of claim 21, where the one or more channels include a swirl chamber inside the delivery tip and adjacent to the outlet.

26. The delivery tip of claim 25, where the delivery tip is characterized by a central axis from the inlet to the outlet, and the swirl chamber is configured such that it intersects a plane that is perpendicular to the central axis in a profile having three sides each defined by a line, the three lines forming a substantially equilateral triangle.

27. The delivery tip of claim 21, where the delivery tip includes a conduit, and the central flow-restricting portion is coupled to but not integral with the conduit.

28. The delivery tip of claim 27, where the one or more channels comprise at least some recesses in the central flow-restricting portion.

29. The delivery tip of claim 27, where the one or more channels comprise at least some recesses in the conduit.

30. The delivery tip of claim 21, further comprising an attachment mechanism configured to couple the delivery tip to the syringe assembly.

31. The delivery tip of claim 30, where the attachment mechanism comprises one or more flanges that are integral with the delivery tip.

32. A delivery tip comprising:
a conduit portion having an inlet and a distal portion comprising a nozzle that includes an outlet;
a central flow-restricting portion disposed inside the conduit portion; and
one or more channels adjacent the central flow-restricting portion that provide fluid communication between the inlet and the outlet, the one or more channels including a section that tapers inwardly toward the outlet.

33. The delivery tip of claim 32, where the central flow-restricting portion is coupled to but not integral with the conduit portion.

34. The delivery tip of claim 32, where the one or more channels include multiple sections, each of which converges at an angle less than 90 degrees.

35. The delivery tip of claim 34, where the one or more channels include three sections, each of which converges at an angle less than 90 degrees.

36. The delivery tip of claim 32, where the one or more channels include a swirl chamber inside the delivery tip and adjacent to the outlet.

37. The delivery tip of claim 36, where the delivery tip is characterized by a central axis from the inlet to the outlet, and the swirl chamber is configured such that it intersects a plane that is perpendicular to the central axis in a profile having three sides each defined by a line, the three lines forming a substantially equilateral triangle.

38. The delivery tip of claim 32, where the delivery tip includes a conduit, and the central flow-restricting portion is coupled to but not integral with the conduit.

39. The delivery tip of claim 38, where the one or more channels comprise at least some recesses in the central flow-restricting portion.

40. The delivery tip of claim 38, where the one or more channels comprise at least some recesses in the conduit.

41. The delivery tip of claim 32, further comprising an attachment mechanism configured to couple the delivery tip to the syringe assembly.

42. The delivery tip of claim 41, where the attachment mechanism comprises one or more flanges that are integral with the delivery tip.

43. A kit comprising:
an adhesive compound comprising a plurality of dispensable components;
a syringe assembly defining two or more reservoirs each configured to hold one of the dispensable components; and
a delivery tip having an inlet, an outlet, a central flow-restricting portion, and one or more channels adjacent the central flow-restricting portion that provide fluid communication between the inlet and the outlet, the one or more channels including a section that tapers inwardly toward the outlet;
where the delivery device is configured such that if the two or more reservoirs hold dispensable components then the syringe assembly can be actuated to dispense the dispensable components from the two or more reservoirs substantially simultaneously into the inlet.

44. The kit of claim 43, where the one or more channels include multiple sections, each of which converges at an angle less than 90 degrees.

45. The kit of claim 44, where the one or more channels include three sections, each of which converges at an angle less than 90 degrees.

46. The kit of claim 43, where the one or more channels include a swirl chamber inside the delivery tip and adjacent to the outlet.

47. The kit of claim 46, where the delivery tip is characterized by a central axis running from the inlet to the outlet, and the swirl chamber is configured such that it intersects a plane that is perpendicular to the central axis in a profile having three sides each defined by a line, the three lines forming a substantially equilateral triangle.

48. The kit of claim 43, where the delivery tip includes a conduit, and the central flow-restricting portion is coupled to but not integral with the conduit.

49. The kit of claim 48, where the one or more channels comprise at least some recesses in the central flow-restricting portion.

50. The kit of claim 48, where the one or more channels comprise at least some recesses in the conduit.

51. The kit of claim 43, further comprising an attachment mechanism configured to couple the delivery tip to the syringe assembly.

52. The kit of claim 51, where the attachment mechanism comprises one or more flanges that are integral with the delivery tip.

53. A kit comprising:
an adhesive compound comprising a plurality of dispensable components;
a syringe assembly defining two or more reservoirs each configured to hold a dispensable component; and
a delivery tip having an inlet, a central flow-restricting portion, a distal portion comprising a nozzle that includes an outlet, and one or more channels adjacent the central flow-restricting portion that provide fluid communication between the inlet and the outlet, the delivery tip being characterized by a central axis running from the inlet to the outlet, the nozzle including a section that tapers outwardly and away from the outlet along the central axis;
where the delivery device is configured such that if the two or more reservoirs hold dispensable component then the syringe assembly can be actuated to dispense the dispensable component from the two or more reservoirs substantially simultaneously into the inlet.

54. The kit of claim 53, where the one or more channels include multiple sections, each of which converges at an angle less than 90 degrees.

55. The kit of claim 54, where the one or more channels include three sections, each of which converges at an angle less than 90 degrees.

56. The kit of claim 53, where the one or more channels include a swirl chamber inside the delivery tip and adjacent to the outlet.

57. The kit of claim 56, where the delivery tip is characterized by a central axis running from the inlet to the outlet, and the swirl chamber is configured such that it intersects a plane that is perpendicular to the central axis in a profile having three sides each defined by a line, the three lines forming a substantially equilateral triangle.

58. The kit of claim 53, where the delivery tip includes a conduit, and the central flow-restricting portion is coupled to but not integral with the conduit.

59. The kit of claim 58, where the one or more channels comprise at least some recesses in the central flow-restricting portion.

60. The kit of claim 58, where the one or more channels comprise at least some recesses in the conduit.

61. The kit of claim 53, further comprising an attachment mechanism configured to couple the delivery tip to the syringe assembly.

62. The kit of claim 61, where the attachment mechanism comprises one or more flanges that are integral with the delivery tip.

63. A method to seal a surgical site or a wound comprising the step of applying an adhesive that is admixed in a delivery device comprising:
a syringe assembly defining a first reservoir and a second reservoir, each configured to hold a dispensable material, wherein the first reservoir contains a solution of a polymeric catechol material and the second reservoir contains a crosslinking solution of a periodate; and
a delivery tip having an inlet, an outlet, a central flow-restricting portion, and one or more channels adjacent the central flow-restricting portion that provide fluid communication between the inlet and the outlet, the one or more channels including a section that tapers inwardly toward the outlet;
where the delivery device is configured such that if the two or more reservoirs hold dispensable material then the syringe assembly can be actuated to dispense the dispensable material from the two or more reservoirs substantially simultaneously into the inlet.
